(19) United States
(12) Patent Application Publication
(10) Pub. No.: US 2010/0112652 A1
(43) Pub. Date: May 6, 2010

(54) Method for Producing Lactic Acid By The Fermentation of a Self-Sufficient Medium Containing Green Cane Juice

(75) Inventors: Jean-Christophe Bogaert, Escanaffles (BE); Philippe Coszach, Escanaffles (BE); Pierre-Antoine Mariage, Escanaffles (BE)

Correspondence Address: BAKER & DANIELS LLP, 300 NORTH MERIDIAN STREET, SUITE 2700, INDIANAPOLIS, IN, 46204, US

(73) Assignee: GALACTIC S.A., Escanaffles (BE)

(21) Appl. No.: 12/524,324

(22) PCT Filed: Jan. 24, 2008
(86) PCT No.: PCT/EP2008/050824

§ 371 Date: Jul. 23, 2009

(30) Foreign Application Priority Data
Jan. 31, 2007 (EP) 07101464.1

(51) Int. Cl. C12P 7/56

(52) U.S. Cl. 435/139

(57) ABSTRACT

The invention relates to a process for producing lactic acid by fermentation of a sugarcane extract by means of microorganisms belonging to the Bacillus or Sporolactobacillus genus. The fermentation medium is self-sufficient.

The process may comprise at least one purification step. The lactic acid produced is heat-stable and is of a particularly high purity.

### FIELD OF THE INVENTION

Lactic acid or 2-hydroxypropanoic acid is an α-hydroxylated carboxylic acid which can be produced by fermentation. Other pathways for obtaining lactic acid are known to those skilled in the art, via chemical conversions of reactants derived from petrochemistry, such as the hydrolysis of lactonitrile, itself obtained starting from acetaldehyde, the chlorination and hydrolysis of propionic acid, or else via the nitration of propene.

Lactic acid exists in two diastereoisomeric forms: L(+) and D(−) lactic acid, for which each day there are new applications, from the conventional use as a food preservative to new developments such as the synthesis of solvents, pesticides, herbicides, biodegradable polymers, etc.

However, due to the increasing strengthening of the quality criteria required and the need to achieve production costs compatible with the commodities market, it is essential to be able to reduce the costs of the starting materials while at the same time ensuring a quality compatible with the most demanding applications.

### PRIOR ART

The purity of a lactic acid is, inter alia, evaluated by means of a thermal stability test consisting in measuring the colour (APHA scale in Hazen units) of the product after refluxing at 200° C. for 2 h. If the lactic acid cooled to ambient temperature after this test has a colour less than 50 Hazen, it will be considered to be heat-stable. However, it is not uncommon to encounter on the market specific applications requiring a colour after heating of less than 20 Hazen, or even less than 10 Hazen.

Lactic acid, for example, used as a starting material for the production of polylactic acid must have a very high purity and a very low thermostability index of the order of 0-50 Hazen, and preferably of 0-20 Hazen. It corresponds to a “polymer” grade.

The production of lactic acid by fermentation can be carried out by adding a lactic acid-producing microorganism to a medium containing a source of purified fermentable carbon, mineral salts (source of nitrogen, of phosphate, of sulphur and of trace elements such as zinc, magnesium, manganese, etc.) and a source of organic nitrogen composed of free amino acids or amino acids bound in the form of oligopeptides and peptides, of vitamins and of traces of enzyme cofactors. It is also known that the microorganisms normally used, of the Lactobacillus, Bacillus and Sporolactobacillus genera, cannot grow and produce lactic acid industrially without the addition of such an organic nitrogen source, whether its composition is defined or whether it is a natural extract.

Examples of carbonaceous substrates which meet these specifications are purified beet sugar and cane sugar and refined glucose syrups originating from the hydrolysis of maize starch, wheat starch, potato starch, and the like.

Examples of organic nitrogen sources which meet the abovementioned specifications are yeast autolysates and hydrolysates, plant protein hydrolysates (soybean tryptone, gluten peptone, etc.) and animal protein hydrolysates (caseine peptone, etc.) and also the soluble by-products from steeping wheat and maize. These organic nitrogen sources are sold at relatively high prices, which puts a great strain on the manufacturing costs of lactic acid. Defined organic nitrogen sources, reconstituted from purified amino acids, vitamins and growth promoters, can also be used, but with even greater expense.

In order to reduce production costs, lactic acid by fermentation can also be obtained by adding a microorganism to a source of inexpensive unrefined fermentable sugars, consisting of intermediates or of by-products from the agricultural industry (starch syrup, lactoserum, raw cane juice, molasses, hydrolysed cane bagasse, etc.), to which is added organic nitrogen, such as soluble by-products from steeping wheat and/or yeast extracts.

Organic impurities are present in large amounts in these sources of unrefined sugars. This amount of organic impurities (other than carbohydrates) can be measured by analyzing the amount of organic nitrogen of a sample. Said amount is expressed in g/kg and is measured by the Kjeldhal method.

These organic impurities may be of plant origin or else may be the result of caramelization reactions or of Maillard reactions involved during the various steps for processing the plant. They may be alcohols, organic acids, aldehydes, sugar degradation products (furans, pyrones, cyclopentenes, organic acids, aldehydes, sulphur compounds, pyrroles, pyridines, imidazoles, pyrazines, oxazoles, thiazoles, etc.), proteins and vitamins.

However, in these sources of unrefined sugars, some of the impurities, such as furfurals and hydroxymethyl-furfurals, are known to inhibit the growth of certain microorganisms and to slow down bioconversion processes such as lactic fermentation. Thus, Payot et al. (1998) have shown that beet molasses contain compounds that inhibit the growth of Bacillus coagulans.

Mention is made of the use of a medium based on raw cane juice and yeast extracts (3 g/l) for the production of lactic acid by fermentation by genetically modified wine yeasts. However, these yeasts are not homofermentative and have a carbon yield (lactic acid produced relative to sugar consumed) of 61%.

The industrial purification of a thermostable-grade lactic acid from a fermentation liquor rich in lactic acid can be carried out by means of various technologies which in general include common steps:
clarification of the fermentation must (centrifugation, flocculation/filtration, micro-filtration, etc.);
elimination of ions (electrodialysis, resins, liquid/liquid extraction, etc.);
elimination of the colour and other impurities (nanofiltration, active carbon, etc.);
concentration/distillation of the lactic acid, these steps having to be coupled so as to obtain a high yield;
other techniques can also be used, such as crystallization.

Some of the impurities present in the sources of unrefined sugars have molecular weights and vapour pressure curves close to that of lactic acid (such as 5-hydroxymethylfurfural), thereby making them difficult to separate from lactic acid. Others are organic acids and may not be efficiently separated from lactic acid by conventional processes such as, for example, electrodialysis, crystallization or liquid-liquid extraction.

It is thus recognized that the industrial production of heat-stable lactic acid—and especially of “polymer” grade lactic acid, should be carried out from a fermentation medium containing a low content of impurities. The presence of organic impurities such as organic acids, aldehydes and alcohols in the fermentation liquor makes the lactic acid difficult and expensive to purify.

An example of the difficulty of producing a lactic acid of heat-stable quality from a source of unrefined carbonaceous substrate is given by document WO 2006/001034 A2. Said document describes the production of polylactic acid by fermentation of inexpensive carbon sources derived from agriculture, such as cane molasses, by strains of the Lactobacillus genus. In order to obtain growth and sufficient productivity, an exogenous organic nitrogen source (soluble by-products from maize steeping and yeast paste) is added to the medium. The process clearly describes that the purified lactic acid obtained from sugarcane molasses is brown in colour. The purification is carried out after formation of the cyclic dimer of lactic acid (the lactide) using a concentrated impure lactic acid solution. This results in the formation of numerous by-products originating from reactions of the impurities with one another and with the lactic acid. In order to purify this lactide, the document describes recrystallization in a solvent based on ethyl acetate and does not mention a yield for recovery of purified lactide. Now, it is known to those skilled in the art that lactide in its L, D and meso forms is partially soluble in ethyl acetate, resulting in a considerable loss of product and poor production yields.

During the production of sugar from the cane, the sugar is first extracted from the cane by mechanical extraction or diffusion. The juice extracted in this step is referred to below as “raw cane juice”. This raw cane juice is subsequently carbonated and filtered in order to extract the insoluble impurities and the organic anions. The filtrate is finally concentrated by evaporation and gives a sugar syrup which is referred to below as “raw cane syrup”. This syrup may be inverted (hydrolysis of the sucrose to glucose and fructose) to give “raw invert cane syrup” or crystallized directly to give what is referred to below as “raw cane sugar” or may be purified and crystallized to give, firstly, the “refined cane sugar” and a by-product loaded with impurities, “the cane molasses”.

The sugar may be produced from the beet by a similar process. The sugar is first extracted from the beet by mechanical extraction or diffusion. The juice extracted in this step is referred to below as “beet diffusion juice”. This juice is then carbonated and filtered in order to extract the insoluble impurities and the organic anions. The filtrate is then concentrated by evaporation and gives a sugar syrup which is referred to below as “raw beet syrup”. This syrup may be crystallized directly and give what is referred to below as “raw beet sugar” or may be purified and crystallized to give, firstly, the “refined beet sugar” and a by-product loaded with impurities, “the beet molasses”.

Thus, up until now, it was accepted by those skilled in the art that the industrial production of a heat-stable lactic acid from sugar required the use of a medium prepared from purified sugar, from a nitrogenous organic substrate and from minerals.

### BRIEF DESCRIPTION OF THE INVENTION

All the above means that, up until now, the production of lactic acid of heat-stable grade corresponding to the quality requirements of the market (food, pharmaceutical, cosmetic) required a lactic fermentation to be carried out on a medium:
of complex composition and preparation comprising many ingredients to be mixed in very precise amounts and requiring the involvement of a qualified preparer;
of generally high cost (purified sugar, rich organic nitrogen source, pure mineral nutrients).

In the interests of developing a process which makes it possible to satisfy the practical and economic constraints more successfully, the applicant has noted, surprisingly, that this objective can be achieved by means of a process consisting of a lactic fermentation, with microorganisms of the Bacillus and/or Sporolactobacillus genus, of a self-sufficient medium prepared from raw cane juice without the addition of other organic and inorganic nutrients, or else from a medium composed of raw cane juice derivatives, rich in nitrogenous organic substances (such as raw cane syrup) without the addition of other organic nutrients.

A fermentation medium which comprises, as carbohydrate source, a plant extract or a plant extract derivative is considered to be self-sufficient for the fermentation of a microorganism if it allows the latter to grow and produce its metabolites without the addition of organic nutrients other than those present in the plant extract or the plant extract derivative used as carbohydrate source.

The lactic acid produced by fermentation can then be purified by the techniques described in the prior art (concentration, distillation, crystallization, ion exchange, etc.) in order to produce a heat-stable lactic acid.

### DETAILED DESCRIPTION OF THE INVENTION

After long periods of research carried out on numerous carbonaceous substrates and numerous lactic acid-producing microbial strains, the applicant has discovered, surprisingly, that raw cane juice and its derivatives (raw cane syrup, raw invert cane syrup, concentrated raw cane juice, dry raw cane juice, etc.):
naturally contain the sugars and the organic growth and productivity promoters required by certain industrial microorganisms belonging to the Bacillus and/or Sporolactobacillus genera for lactic fermentation;
do not contain compounds capable of inhibiting the growth of microorganisms of the Bacillus and/or Sporolactobacillus genera;
do not contain impurities that prevent the production of a highly pure lactic acid by means of purification processes, and more particularly those comprising an evaporation and/or a distillation and/or crystallization and/or ion exchange;
that the raw cane juice also contains the mineral salts required for the growth of the microorganism.

These unexpected properties of the raw cane juice and its derivatives containing nitrogenous organic substances (raw cane syrup, raw invert cane syrup, concentrated raw cane juice, dry raw cane juice, etc.) are exploited in this invention, which describes an original process for producing a heat-stable lactic acid from these media.

These discoveries are surprising given that our research studies also showed that the unrefined beet derivatives were not self-sufficient and therefore required the addition of an organic nitrogenous substrate in order for certain microorganisms belonging to the Bacillus and Sporolactobacillus genera for lactic fermentation to be able to achieve productivities equivalent to those observed in a conventional industrial medium. These observations are described in the examples.

Furthermore, as also described in the examples, the raw cane juice and its derivatives containing nitrogenous organic substances do not constitute self-sufficient media for all lactic acid-producing microorganisms.

These discoveries are all the more surprising since, in addition to being a self-sufficient medium for certain microorganisms of the Bacillus and/or Sporolactobacillus genera, the medium at the end of fermentation can be purified, by conventional evaporation, distillation, crystallization and/or ion exchange techniques, to a heat-stable lactic acid of “polymer” grade which corresponds to the quality requirements of the market (food, pharmaceutical, cosmetic, industrial). This is not, for example, the case for sugarcane molasses.

The originality shown by the applicant lies in the choice of this starting material as source of at the same time inexpensive sugar, minerals and organic nitrogen, unlike other research studies carried out on inexpensive sugars such as molasses, to which an exogenous organic nitrogen source such as yeast extracts is added.

The use of the liquid or solid, crude or purified raw cane juice as self-sufficient fermentation medium as described above makes it possible to drastically reduce the lactic acid production costs and facilitates the preparation of the fermentation media.

The use of crude or clarified raw cane juice makes it possible to avoid the use of energy for evaporating the water during the concentration or crystallization thereof, and thus to considerably reduce the consumption of energy and the environmental pollution.

The process also makes it possible to prevent carbohydrate losses due to the purification of the sugarcane (principally molasses). The overall lactic acid production yield from the sugarcane is thus greater. In fact, one tonne of sugarcane contains 150 to 180 kg of sugar, but the current production processes make it possible to produce only approximately 120 kg of purified cane sugar, i.e. approximately 120 kg of lactic acid after fermentation. The direct use of the raw cane juice or of its derivatives will allow the use of virtually all the sugar present in the sugarcane and therefore will make it possible to produce between 150 and 180 kg of lactic acid after fermentation, i.e. an increase in the amount of lactic acid produced of 20 to 30%.

The process also makes it possible to eliminate the raw cane juice clarification step. This step consists in precipitating the organic impurities with lime, thereby forming insoluble calcium salts. After numerous research studies, we have discovered that the step of separating the biomass either by flocculation, by microfiltration, by nanofiltration or by any other technique means that there is no need to clarify the raw cane juice. This greatly simplifies the overall process for producing lactic acid from sugarcane.

Other details and particularities of the invention, given hereinafter by way of nonlimiting examples, are apparent from the description as some possible embodiments of said invention.

### Example 1

### Production of a Dry Raw Cane Juice

The dry raw cane juice can be produced, for example, by the following technique. The raw cane juice is extracted from the cane by mechanical extraction in a mill. The juice is then filtered through 10 μm in order to extract the insoluble impurities.

The composition of the raw juice obtained is given in Table 1.

TABLE 1
Composition of the raw cane juice

| | Average content |
|---|---|
| | in g/l |
| Sucrose | 160 |
| Glucose | 5.7 |
| Fructose | 5.7 |
| Proteins | 0.95 |
| Starch | 0.12 |
| Gums | 1 |
| Waxes | 0.24 |
| K₂O | 1 |
| CaO | 0.4 |
| MgO | 0.53 |
| P₂O₅ | 0.76 |
| SiO₂ | 6.7 |
| SO₃ | 1 |
| Organic acids (aconitic acid, citric | 5.7 |
| acid, etc.) | |
| Various (fibres, colloids, | 30 |
| chlorophyll, polyphenols, etc.) | |

The filtrate (18% solids) is then concentrated in an evaporator at 70° C. and at 700 mbar in order to attain 85% solids.

The raw cane juice concentrate can be conveyed while hot to an atomizer at 150° C. at 125 mbar or can be mixed with 5% dry raw cane juice (as nucleation support) and conveyed while hot to a drum dryer at 150° C. at 125 mbar, so as to give a dry product.

### Example 2

### Culturing of Bacillus coagulans on Various Sugar Sources

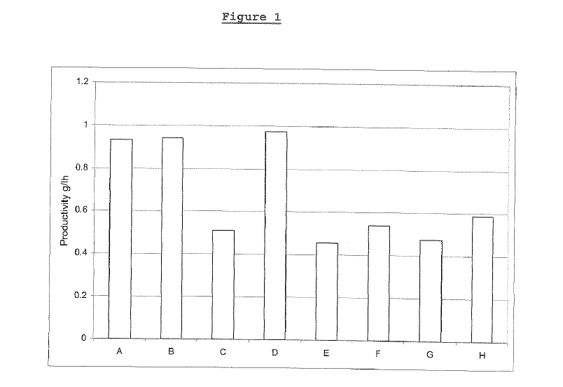

A culture of Bacillus coagulans (LMG 17452) was cultured in BBraun 2 l Biostat B reactors on one of the fermentation media described in Table 2, at 52° C. and maintained at pH 6.2 with 25% by weight Ca(OH)₂ milk. The culture was maintained routinely by transferring 250 ml of the culture every 24 h into a new fermenter containing 750 ml of medium. The rate of lactic acid production or productivity in gram per litre and per hour was followed for 5 fermentations in a row on 8 media with different compositions (in FIG. 1, each column is representative of the mean of the results of the 5 trials).

In FIG. 1, it can clearly be seen that the fermentations carried out on the media B (dry raw cane juice without salts and without yeast extract) and D (raw cane syrup without yeast extract but with salts) have lactic acid productivities similar to the fermentations on medium A (white sugar with yeast extract and salts).

Furthermore, the results show unambiguously that the fermentations on media C (raw cane syrup without yeast extract and without salts) and also the fermentations on media E and F (beet diffusion juice) and G and H (raw beet syrup) give productivities lower than the fermentations carried out on media A, B and D.

TABLE 2
Composition of the culture media tested in Example 2

| Ingredients in g/l | A | B | C | D | E | F | G | H |
|---|---|---|---|---|---|---|---|---|
| Sucrose | 180 | 0 | 0 | 0 | 0 | 0 | 0 | 0 |
| Dry raw cane juice | 0 | 200 | 0 | 0 | 0 | 0 | 0 | 0 |
| Raw cane syrup | 0 | 0 | 300 | 300 | 0 | 0 | 0 | 0 |
| Dry beet | 0 | 0 | 0 | 0 | 200 | 200 | 0 | 0 |
| diffusion juice | | | | | | | | |
| Raw beet syrup | 0 | 0 | 0 | 0 | 0 | 0 | 300 | 300 |
| Yeast extract | 5 | 0 | 0 | 0 | 0 | 0 | 0 | 0 |
| K₂HPO₄ | 0.5 | 0 | 0 | 0.5 | 0 | 0.5 | 0 | 0.5 |
| KH₂PO₄ | 0.5 | 0 | 0 | 0.5 | 0 | 0.5 | 0 | 0.5 |
| MgSO₄•7H₂O | 0.2 | 0 | 0 | 0.2 | 0 | 0.2 | 0 | 0.2 |
| MnCl₂•4H₂O | 0.03 | 0 | 0 | 0.03 | 0 | 0.03 | 0 | 0.03 |
| (NH₄)Cl | 1 | 0 | 0 | 1 | 0 | 1 | 0 | 1 |
| FeCl₃•6H₂O | 0.01 | 0 | 0 | 0.01 | 0 | 0.01 | 0 | 0.01 |

### Example 3

### Culturing of Bacillus coagulans on Dry Raw Cane Juices of Different Geographical Origins

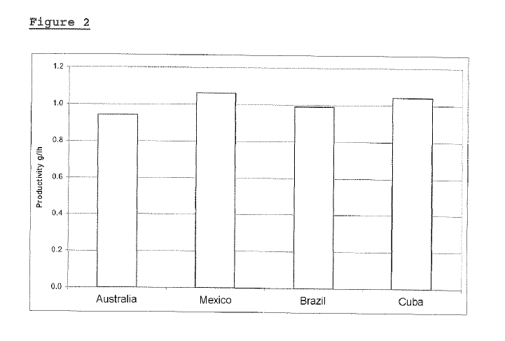

In order to establish whether the geographical origin of the raw cane juice had an influence on the lactic acid productivity of Bacillus coagulans (LMG 17452), we cultured this bacterium on a medium consisting only of water and dry raw cane juices of various origins (Australia, Mexico, Brazil, Cuba) at 50 g/l at 52° C. and maintained at pH 6.2 with 25% by weight Ca(OH)₂ milk. The culture was maintained routinely by transferring 250 ml of the culture every 24 h into a new fermenter containing 750 ml of medium. The lactic acid production rate was followed for 5 fermentations in a row (in FIG. 2, each column is representative of the mean of the results of the 5 trials).

In FIG. 2, the results clearly show that the lactic acid productivities are similar (between 0.9 g/lh and 1.1 g/lh) irrespective of the geographical origin of the sugarcane.

### Example 4

### Purification of the Lactic Acid Produced From Raw Cane Syrup with Salts

### Fermentation

Bacillus coagulans (LMG 17452) was cultured in a BBraun 50 l reactor on a fermentation medium (identical to medium D of Example 2, but a carbonaceous substrate concentration of 75 g/l instead of 300 g/l) consisting of raw cane syrup (65% with respect to sucrose) with salts at 52° C. and maintained at pH 6.2 with 25% by weight Ca(OH)₂ milk. The results are given in Table 3.

TABLE 3
Results of the fermentation

| | Final lactic | Final | Yield of lactic |
|---|---|---|---|
| Lactic acid | acid | volume/initial | acid produced |
| productivity | concentration | volume ratio | relative to |
| g/lh | g/l | (dilution with lime) | initial sucrose (g/g) |
| 1 | 44 | 1.05 | 0.95 |

The fermentation liquor obtained was purified according to the process described below.

### Low Concentration Purification

After having flocculated the biomass and having filtered it, the clarified liquor is acidified by gradual addition of concentrated sulphuric acid so as to precipitate the calcium in the form of CaSO₄. The CaSO₄ is then separated via filtration.

The liquor is then pre-purified on an active carbon column. The percolate is fed onto an ion exchange column, packed with a strong cationic resin (of the Bayer Lewatit S 2528 type). Once the decationization has been carried out, the liquor is fed onto a column packed with an anionic resin of average basicity (of the Bayer type under the reference Lewatitt S 4328). The lactic acid obtained then has the following characteristics:
APHA colour: 200 Hazen
Lactic acid content: 45 g/l
Cation content: <50 meq/1
Anion content: <50 meq/1.

### High Concentration Purification

The liquor purified above is 80% concentrated on a falling-film evaporator before being fed continuously into a mechanically stirred, thin-film borosilicate glass evaporator with an internal condenser. The concentration parameters are 100° C. for the walls and an absolute pressure of 100 mbar.

Finally, the lactic acid is distilled at 10 mbar and 130° C. on this same evaporator.

The lactic acid produced by this purification corresponds to the characteristics (Table 4) of a heat-stable lactic acid. In fact, the fresh solution has a colour of 11 Hazen and its colour after heating reaches 15 Hazen.

The lactic acid produced by this purification corresponds to the specifications of a highly pure lactic acid that can be used for the manufacture of lactide and of polylactic acid.

TABLE 4
Characteristics of the purified lactic acid obtained from a solution
of lactic acid originating from the fermentation by Bacillus coagulans
(LMG 17452) of a medium composed of raw cane syrup

| Lactic acid content | % weight | 90.5 |
|---|---|---|
| L-Lactic acid stereochemical | % L/(% L + % D) | 99.5% |
| purity | | |
| Colour (fresh solution) | Hazen | 11 |
| Colour after heating (200° C., | Hazen | 15 |
| 2 h) | | |

In order to demonstrate that the lactic acid produced was suitable for the production of polylactic acid, we initiated the following trials.

### Synthesis of Lactide and Polymerization Test

The lactic acid obtained above (˜250 g) is introduced into a round-bottomed flask stirred and heated to 160° C. In order to facilitate the rapid extraction of the volatile compound, the unit is gradually placed under vacuum, the pressure ranging between atmospheric pressure and 150 mbar for approximately 10 h. The lactic acid polymerizes so as to form a prepolymer characterized by a molecular mass of 1500 daltons.

The prepolymer obtained above is introduced into a round-bottomed flask heated by means of a heating cap to 220-250° C. and stirred by means of a magnetic chip. Tin octoate is then introduced into the round-bottomed flask at 1% by weight relative to the amount of prepolymer introduced.

The round-bottomed flask is surmounted by a reflux condenser at 180-200° C., and then by a condenser cooled to 80-100° C. and, finally, by a round-bottomed flask for harvesting the condensates. The whole is placed under a vacuum of between 10 and 20 mbar. The impure lactide harvested in the round-bottomed condensate flask is purified twice by recrystallization in a 1:1 ratio with toluene.

The crystals of purified lactide are recovered by filtration and dried under vacuum in a rotary evaporator.

The lactide purified in this manner has the following characteristics:
L-lactide: 99.8%
meso-lactide: 0.2%
residual acidity: <10 meq/kg
water content: 49 ppm.

A small amount of the purified product obtained above (10 g) was introduced into a test tube under flushing with nitrogen (several trials were initiated in parallel). After solubilization of the mixture (100° C.), a solution of tin octoate was added in such a way as to observe a dimer/catalyst molar ratio of 4500. Once the solution was well homogenized, it was immersed in a bath of oil, the temperature of which was thermostatted at 180° C.

After synthesis for one hour, the test tubes were removed and broken so as to recover polymers that were very rigid and opaque. The polymers obtained were analyzed by GC in chloroform at 35° C. and number-average molecular masses of between 80 000 and 100 000 were measured (the molecular masses determined on the basis of a PS calibration are corrected on an absolute basis using a universal calibration).

This example therefore shows that it is possible to achieve a lactic acid of “polymer” quality using raw cane syrup as carbonaceous fermentation substrate.

### Example 5

### Purification of the Lactic Acid Produced From Sugarcane Molasses

In Example 2, we showed that, unlike beet derivatives, the cane derivatives made it possible, in the form of a self-sufficient medium, to obtain a lactic fermentation comparable to conventional industrial media. In this example, we will show that certain cane derivatives, although self-sufficient, do not make it possible to produce a lactic acid of heat-stable quality.

Bacillus coagulans (LMG 17452) was cultured in a BBraun 50 l reactor on a fermentation medium (identical to medium D of Example 2 with a carbonaceous substrate concentration of 75 g/l instead of 300 g/l) consisting of sugarcane molasses (60% with respect to sucrose) with salts, at 52° C. and maintained at pH 6.2 with 25% by weight Ca(OH)₂ milk. The results are given in Table 5.

TABLE 5
Results of the fermentation

| | Final lactic | Final | Yield of lactic |
|---|---|---|---|
| Lactic acid | acid | volume/initial | acid produced |
| productivity | concentration | volume ratio | relative to |
| g/lh | g/l | (dilution with lime) | initial sucrose (g/g) |
| 1 | 40 | 1.05 | 0.94 |

The fermentation liquor obtained was purified according to the same procedure as Example 4.

The following product was obtained after low concentration purification:
APHA colour: 200 Hazen
Lactic acid content: 45 g/l
Cation content: <50 meq/1
Anion content: <50 meq/1

On the other hand, surprisingly, the lactic acid produced by the high concentration purification does not correspond to the specifications of a highly pure lactic acid that can be used for the manufacture of lactide and of polylactic acid. In fact, the fresh solution already has a colour of 70 Hazen and its colour after heating reaches 230 Hazen.

It is therefore surprising that, in the case of Example 4, the use of raw cane syrup as source of carbon and of organic nitrogen for the fermentation makes it possible to obtain a highly pure (thermostable) lactic acid.

TABLE 6
Characteristics of the purified lactic acid obtained from a solution
of lactic acid originating from the fermentation with Bacillus coagulans
(LMG 17452) of a medium composed of sugarcane molasses

| Lactic acid content | % weight | 90.2 |
|---|---|---|
| Colour (fresh solution) | Hazen | 70 |
| Colour after heating (200° C., 2 h) | Hazen | 230 |

### Example 6

### Test of Fermentation of Raw Cane Syrup with Other Microorganisms

Various strains were cultured in BBraun 2 l reactors on a fermentation medium (see Example 4) consisting of raw cane syrup (65% with respect to sucrose) with salts at 52° C. and maintained at pH 6.2 with 25% by weight Ca(OH)₂ milk. The results are given in Table 7.

In Table 7 below, it can be seen that the cane syrup is not a self-sufficient medium for all the lactic acid-producing microorganisms, but only for some of them, including certain species of Bacillus and Sporolactobacillus.

TABLE 7
Fermentation of the raw cane syrup starting
with lactic acid-producing microorganisms

| | | Produc- | | Final |
|---|---|---|---|---|
| | | tivity | Stereo-chemical | lactic acid |
| | Origin | (g/lh) | purity (% of L) | produced (g) |
| Bacillus | LMG 17452 | 1 | 99 | 45 |
| coagulans | | | | |
| Bacillus | LMG 19808 | 1 | 99 | 40 |
| coagulans | | | | |
| Sporolacto- | DSM 20348 | 0.5 | 1 | 20 |
| bacillus | | | | |
| inulinus | | | | |
| Bacillus | DSM 459 | 0.5 | 97 | 20 |
| smithii | | | | |
| Lactobacillus | DSM 2601 | 0 | — | 0 |
| plantarum | | | | |
| Lactobacillus | DSM 20004 | 0 | — | 0 |
| coryniformis | | | | |

### Example 7

### Improving Fermentation by Enriching the Fermentation Medium with Yeast Extract

As described in the examples above, a self-sufficient fermentation medium prepared from raw cane juice and fermented with Bacillus coagulans (LMG 17452) allows a lactic acid productivity comparable to complex media prepared from purified sugars, minerals and an organic nitrogen source.

We have observed that this productivity could be further improved (+20%) by adding an exogenous organic nitrogen source (yeast extract at 0.5 g/l) to this medium prepared from raw cane juice.

The invention relates to a process for producing lactic acid by fermentation of a sugarcane extract or of sugarcane extract derivatives by means of microorganisms. This process is characterized by the fermentation microorganisms which belong to the Bacillus and Sporolactobacillus genera, or mixtures thereof, and by the fermentation medium which is self-sufficient.

This process preferably comprises at least one step of purifying the lactic acid derived from the fermentation. The purification preferably comprises at least one step chosen from evaporation, distillation, crystallization or the use of ion exchange resins.

The microorganisms are automatically chosen from the species Bacillus coagulans, Bacillus smithii and Sporolactobacillus inulinus, or mixtures thereof.

The process produces a heat-stable lactic acid.

The sugarcane or the sugarcane derivative advantageously has a concentration in terms of organic nitrogen of greater than 0.02 g/kg of fermentation medium. The sugarcane extract is preferably chosen from the raw cane juice, the raw cane syrup, the raw invert cane syrup, the raw cane sugar, or derivatives thereof. The sugarcane extract or the sugarcane extract derivatives are preferably in the form of a liquid or in the form of a dry solid.

The sugarcane extract derivative is obtained by a concentration method advantageously chosen from atomization, evaporation, crystallization or centrifugation.

In one embodiment, the fermentation medium is enriched with yeast autolysates and hydrolysates, plant protein hydrolysates or animal protein hydrolysates, or with soluble by-products from steeping wheat or maize.

In one embodiment, the medium is enriched with a purified sugar chosen from glucose, maltose, fructose, xylose or sucrose.

In one embodiment, the sugarcane extract or the sugarcane extract derivatives is (are) sterilized mechanically, thermally or chemically before fermentation.

1. Process for producing lactic acid by fermentation of a sugarcane extract or of sugarcane extract derivatives by means of microorganisms, characterized in that:
a) the microorganisms of the fermentation belong to the Bacillus or Sporolactobacillus genus, or mixtures thereof;
b) the fermentation medium is self-sufficient.

2. Process according to claim 1, characterized in that it comprises at least one step of purifying the lactic acid derived from the fermentation.

3. Process according claim 2, characterized in that the purification comprises at least one step chosen from evaporation, distillation, crystallization and the use of ion exchange resins.

4. Process according claim 1, characterized in that the microorganisms are chosen from the species Bacillus coagulans, Bacillus smithii and Sporolactobacillus inulinus, or mixtures thereof.

5. Process according claim 1, characterized in that the lactic acid produced is heat-stable.

6. Process according claim 1, characterized in that the sugarcane has a concentration in terms of organic nitrogen of greater than 0.02 g/kg of fermentation medium.

7. Process according to claim 1, characterized in that the sugarcane extract is chosen from at least one of raw cane juice, raw cane syrup, raw invert cane syrup, and raw cane sugar.

8. Process according claim 1, characterized in that the sugarcane extract is in the form of a liquid or in the form of a dry solid.

9. Process according claim 1, characterized in that the sugarcane extract derivative is obtained by means of a method of concentration chosen from atomization, evaporation, crystallization or centrifugation.

10. Process according to claim 1, characterized in that the medium is enriched with yeast autolysates and hydrolysates, plant protein hydrolysates or animal protein hydrolysates, or with soluble by-products from steeping wheat or maize.

11. Process according to claim 1, characterized in that the medium is enriched with a purified sugar chosen from glucose, maltose, fructose, xylose or sucrose.

12. Process according to claim 1, characterized in that the sugarcane extract is sterilized mechanically, thermally or chemically before fermentation.

13. Process according to claim 1, characterized in that the sugarcane extract derivative is chosen from one of the derivatives of raw cane juice, raw cane syrup, raw invert cane syrup, and raw cane sugar.

14. Process according claim 1, characterized in that the sugarcane extract derivative is in the form of a liquid or in the form of a dry solid.

15. Process according to claim 11, characterized in that the medium is also enriched with yeast autolysates and hydrolysates, plant protein hydrolysates or animal protein hydrolysates, or with soluble by-products from steeping wheat or maize.

16. Process according to claim 10, characterized in that the medium is also enriched with a purified sugar chosen from glucose, maltose, fructose, xylose or sucrose.

17. Process according to claim 1, characterized in that the sugarcane extract derivatives are sterilized mechanically, thermally or chemically before fermentation.

18. Process according claim 1, characterized in that the sugar cane derivative has a concentration in terms of organic nitrogen of greater than 0.02 g/kg of fermentation medium.
